(19) United States
(12) Patent Application Publication
(10) Pub. No.: US 2022/0218526 A1
(43) Pub. Date: Jul. 14, 2022

(54) METHOD FOR CONTROLLING AN EYE SURGICAL LASER, COMPUTER PROGRAM PRODUCT, AND TREATMENT APPARATUS

(71) Applicant: SCHWIND eye-tech-solutions GmbH, Kleinostheim (DE)

(72) Inventors: Samuel ARBA MOSQUERA, Aschaffenburg (DE); Mario SHRAIKI, Ober-Ramstadt (DE); Nico TRIEFENBACH, Mainaschaff (DE)

(21) Appl. No.: 17/562,316

(22) Filed: Dec. 27, 2021

(30) Foreign Application Priority Data
Jan. 13, 2021 (DE) 10 2021 100 509.4

(51) Int. Cl. A61F 9/008

(52) U.S. Cl. CPC A61F 9/00825; A61F 2009/00878; A61F 2009/00882; A61F 2009/00872

(57) ABSTRACT

A method for controlling an eye surgical laser is disclosed for the separation of a volume body with a predefined posterior interface and a predefined anterior interface. The method includes controlling the laser by means of a control device such that it emits pulsed laser pulses into the cornea. Predefined posterior and anterior interfaces are generated by means of an interaction of the individual laser pulses with the cornea by the generation of cavitation bubbles along a rotation path. A respective interface is divided at least into an inner annulus and an outer annulus, and the cavitation bubbles are generated along the rotation path from an inner boundary of the outer annulus to an outer boundary of the outer annulus. Also disclosed in relation to the method are a computer program, a computer-readable medium and a treatment apparatus.

### FIELD

The invention relates to a method for controlling an eye surgical laser for the separation of a volume body with a predefined posterior interface and a predefined anterior interface from a human or animal cornea and to a method for performing a surgical procedure on a human or animal cornea. Further, the invention relates to a computer program product, to a computer-readable medium as well as to a treatment apparatus.

### BACKGROUND

Opacities and scars within the cornea, which arise by inflammations, injuries or congenital diseases, as well as visual disorder, such as for example myopia or hyperopia, impair the sight. In particular in case that these pathological and/or unnaturally altered areas of the cornea are located in the axis of vision of the eye, clear sight is considerably disturbed. In known manner, the thus altered areas are eliminated by so-called phototherapeutic keratectomy (PTA) by means of an ablatively acting laser, for example an excimer laser. However, this is only possible if the pathological and/or unnaturally altered areas of the cornea are located in the superficial layers of the cornea. Subjacent areas, in particular within the stroma, are not reachable by means of ablative laser methods. Here, additional measures, such as for example the exposure of the subjacent areas, have to be taken by means of an additional corneal incision. By these additional measures, the treatment duration is disadvantageously considerably increased. In addition, there is the risk that further complications, such as for example the occurrence of inflammations, occur at the incision locations by the additional corneal incisions.

Further, it is known from the prior art that a lenticule is generated from an outer area of the lenticule to an inner area of the lenticule by photodisruption and by the generation of a plurality of cavitation bubbles in case of subjacent areas. The generation of the cavitation bubbles from the outer to the inner area is performed in the prior art to prevent an uncontrolled intrastromal gas spreading, so-called opaque bubble layers, within the cornea. The probability of the development of these opaque bubble layers can be reduced in the method of generating the cavitation bubbles from the outside to the inside since excessive energy in the generated cavitation bubble can then be delivered to an adjacent external and already generated cavitation bubble.

However, it is disadvantageous in this method that the external area of the lenticule is first generated at the beginning of the treatment, wherein the area to be treated is often in the inner area of the lenticule, such that the treatment is required in particular there. Thus, the treatment of the internal area is effected only at the end of the actual treatment on the patient such that he can already be exhausted. Further, a predefined adjustment at the laser can be shifted towards the end of the treatment. Further, the cornea of the eye is not yet affected by the treatment at the beginning of the treatment.

### SUMMARY

Therefore, it is the object of the present invention to provide a method and a treatment apparatus for controlling an eye surgical laser for the separation of a volume body with a predefined posterior interface and a predefined anterior interface from a human or animal cornea, and a method for performing a surgical procedure, by which the disadvantages of the prior art are overcome.

This object is solved by a method, a treatment apparatus, a computer program as well as a computer-readable medium according to the independent claims. Advantageous configurations with convenient developments of the invention are specified in the respective dependent claims, wherein advantageous configurations of the method are to be regarded as advantageous configurations of the treatment apparatus, of the computer program and of the computer-readable medium and vice versa.

A first aspect of the invention relates to a method for performing a surgical procedure on a human or animal cornea for the separation of a volume body from the cornea and to a method for controlling an eye surgical laser for the separation of a volume body with a predefined posterior interface and a predefined anterior interface from a human or animal cornea. The laser is controlled by means of a control device such that it emits pulsed laser pulses in a shot sequence into the cornea, wherein the interfaces are generated by means of an interaction of the individual laser pulses with the cornea by the generation of a plurality of cavitation bubbles along a respective rotation path. A respective interface is divided at least into an inner annulus and into an outer annulus, wherein the cavitation bubbles are generated along the rotation path from an inner boundary of the outer annulus, which faces an outer boundary of the inner annulus, to an outer boundary of the outer annulus.

Thereby, a more efficient treatment can be performed with respect to the prior art. In particular, in the prior art, the generation of the cavitation bubbles is performed from the outside, thus an outer area of the volume body, towards the inside, thus an inner area of the volume body, whereby an area of the center is treated only at a later point of time. The inner area is in particular formed in the vicinity of the center and can for example encompass the center. In particular, the corresponding treatment and correction, respectively, are mainly required in the inner area of the volume body. Thus, it is presently provided that the inner area in immediate vicinity to the center of the volume body is first treated and that the laser pulses for the inner area are first generated, respectively. A size of the inner annulus is selected correspondingly small. The outer annulus includes and encompasses, respectively, the inner area at least in certain areas. Thus, at the beginning of the treatment, the inner area is already treated by the generation of the cavitation bubbles at the inner boundary of the outer annulus, wherein the patient is herein in particular still unstressed and fresh. Further, the cornea is also still in an unstressed state. The eye surgical laser is also still correspondingly accurately calibrated and thus does not have any deviations, which can occur during the treatment.

Preferably, the cavitation bubbles are generated by means of photodisruption. Therein, according to the invention, so-called opaque bubble layers can now in particular be prevented in the inner area and a treatment from the inside to the outside can nevertheless be realized such that the disadvantages of the prior art are overcome. An opaque bubble layer is the accumulation of gas bubbles, which are temporarily retained in the intrastromal interface and cause a temporary opacity. Herein, the gas bubbles can expand the stroma because they cannot escape. Thus, it is of crucial importance that these opaque bubble layers are prevented. In the outer annulus, the distances of the cavitation bubbles are correspondingly high such that the development of the opaque bubble layers is prevented there.

In particular, the generation of the cavitation bubbles is rotationally symmetrically effected, in particular spirally from the inside to the outside along the rotation path.

Due to physically preset repetition rates of the treatment apparatus of the generation of the laser pulses and due to the rotational speeds of a beam deflection device of the treatment apparatus, respectively, a reduction of the distances of the cavitation bubbles can only be realized with high effort especially in the center of the lenticule and of the volume body, respectively. According to the invention, it is now provided that the cavitation bubbles are first generated from an inner boundary of the outer annulus to the outer boundary of the outer annulus. Thus, the opaque bubble layers can in particular be prevented at least in the outer annulus.

In particular, the inner annulus has a considerably lower radius with respect to the outer annulus. Therein, the outer annulus surrounds the inner annulus, wherein the outer annulus encompasses a major part of the inner area and of the area to be treated around the center of the volume body. The inner annulus in particular encompasses the center.

According to an advantageous form of configuration, cavitation bubbles are not generated in the inner annulus. In particular, the outer and the inner radius of the inner annulus are selected correspondingly small relative to the radius of the outer annulus. In this form of configuration, the inner annulus can be disregarded with respect to a correction and an incision for separation there. In the extraction of the volume body, the inner annulus is then also separated, although cavitation bubbles have not been generated there. Nevertheless, the cavitation bubbles can be generated from the inside to the outside along the rotation path, whereby a more efficient treatment of the patient can be realized with prevention of the development of opaque bubble layers at the same time.

Further, it has proven advantageous if an inner boundary of the inner annulus is selected with a radius equal to zero. In particular, the inner annulus can then be formed as a disk. For example, the disk in the center of the volume body cannot be provided with cavitation bubbles. Thus, corresponding opaque bubble layers can in particular be prevented in the center. By a corresponding choice of the radius for the outer boundary of the inner annulus, a reliable removal of the volume body from the cornea can nevertheless be realized.

In a further advantageous form of configuration, a respective distance between adjacent cavitation bubbles is increased in the inner annulus with respect to a respective distance between adjacent cavitation bubbles in the outer annulus and/or a respective distance between adjacent rotation path parts of the rotation path is increased in the inner annulus with respect to a respective distance between adjacent rotation path parts of the rotation path in the outer annulus. Thus, the distances between the cavitation bubbles on the rotation path and between the rotation paths, respectively, can in particular be increased in the inner annulus itself, whereby corresponding overlaps and thus opaque bubble layers can be prevented.

Further, it has proven advantageous if a repetition rate for emitting the laser pulses is changed for increasing the distance and/or a rotational speed of a deflection device for the laser of the treatment apparatus is changed for increasing the distance. Thus, it is allowed that the distances between the adjacent cavitation bubbles and on the different rotation path parts, respectively, can be increased in the inner annulus, whereby an overlap of the cavitation bubbles and thus corresponding opaque bubble layers can be reliably prevented.

It is further advantageous if an inner boundary of the inner annulus is selected with a radius greater than zero. Thus, a small disk in particular remains in the center of the volume body before removal of the volume body. In particular, the radius greater than zero can be selected correspondingly low such that a reliable removal of the volume body can nevertheless be realized. In particular, the cavitation bubbles are also generated from an inner boundary to an outer boundary in the inner annulus.

Further, it has proven advantageous if the cavitation bubbles are generated in the inner annulus from the outer boundary of the inner annulus to an inner boundary of the inner annulus, and temporally thereafter, the cavitation bubbles are generated from the inner boundary of the inner annulus to the outer boundary of the inner annulus. Thus, the cavitation bubbles are in particular generated from the outer boundary of the inner annulus towards the center of the volume body. Temporally thereafter, new generation of the cavitation bubbles from the inner boundary of the inner annulus to the outer boundary of the inner annulus is effected. Thus, the cavitation bubbles are generated twice. Thereby, a simple control of the laser can be effected.

In a further advantageous form of configuration, temporally after generating the cavitation bubbles from the inner boundary of the inner annulus to the outer boundary of the inner annulus, the cavitation bubbles are generated from the inner boundary of the outer annulus to the outer boundary of the outer annulus. In other words, it is provided that the cavitation bubbles are first generated in the inner annulus from the inside to the outside. Subsequently thereto, the cavitation bubbles are generated from the inner boundary of the outer annulus from the inside to the outside to the outer boundary of the outer annulus. Thus, an efficient generation of the volume body can be realized.

It is further advantageous if the cavitation bubbles are generated in the inner annulus from the outer boundary of the inner annulus to an inner boundary of the inner annulus, and temporally thereafter, the cavitation bubbles are generated from the inner boundary of the outer annulus to the outer boundary of the inner annulus. Thus, it is in particular provided that the cavitation bubbles are first generated from the outside to the inside in the inner annulus. From the inner boundary of the inner annulus, it is then jumped to the inner area of the outer annulus and the cavitation bubbles are generated there from the inside to the outside. Thus, a reliable separation of the volume body can be realized.

In a further advantageous form of configuration, the cavitation bubbles are generated from the inner boundary of the outer annulus to the outer boundary of the inner annulus in a first time step, and temporally subsequently, the cavitation bubbles are generated from an inner boundary of the inner annulus to the outer boundary of the inner annulus. Thus, the cavitation bubbles are in particular generated in the respective annuli from the inside to the outside. First, it is begun in the so-called periphery, thus in the outer annulus. Subsequently thereto, it is jumped to the inner boundary of the inner annulus and the cavitation bubbles are generated from there to the outer boundary of the inner annulus. Thus, the volume body can be reliably separated without opaque bubble layers being observed.

Further, it has proven advantageous if the respective interfaces are divided at least into an additional middle annulus. In particular, the middle annulus is located between the outer annulus and the inner annulus. In particular, the respective annuli are then generated from the inside to the outside. For example, it can be provided that the cavitation bubbles are first generated from the inner boundary of the outer annulus to the outer boundary of the outer annulus. In a next time step, the generation of the cavitation bubbles is effected from an inner boundary of the middle annulus to an outer boundary of the middle annulus. Subsequently thereto, the cavitation bubbles can in turn be generated from the inner boundary of the inner annulus to the outer boundary of the inner annulus. The interfaces can also be divided into more than three annuli.

It is further advantageous if the control of the laser is effected such that topographic and/or pachymetric and/or morphologic data of the cornea is taken into account. Thus, topographic and/or pachymetric measurements of the cornea to be treated as well as of the type, the position and the extent of the for example pathological and/or unnaturally altered area within the stroma of the cornea as well as corresponding visual disorders of the eye can in particular be taken into account. In particular, control datasets are generated at least by providing topographic and/or pachymetric and/or morphologic data of the untreated cornea and providing topographic and/or pachymetric and/or morphologic data of the pathological and/or unnaturally altered area to be removed within the cornea and considering corresponding optical corrections for removing the visual disorders.

According to a further advantageous form of configuration, the control of the laser is effected such that the laser emits laser pulses in a wavelength range between 300 nanometers and 1400 nanometers, in particular between 700 nanometers and 1200 nanometers, at a respective pulse duration between 1 fs and 1 ns, in particular between 10 fs and 10 ps, and a repetition frequency of greater than 10 kHz, in particular between 100 kHz and 100 MHz. Such lasers are already used for photodisruptive methods in the eye surgery. The produced lenticule, which corresponds to the volume body, is subsequently removed via an incision in the cornea. The use of photodisruptive lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy in the generation of the interfaces is ensured.

A second aspect of the invention relates to a treatment apparatus with at least one eye surgical laser for the separation of a volume body with predefined interfaces of a human or animal eye by means of photodisruption, and with at least one control device for the laser or lasers, which is formed to execute the steps of the method according to the preceding aspect. The treatment apparatus additionally includes a rotation scanner for predefined deflection of the laser beam of the laser towards the eye to be treated. The treatment apparatus according to the invention allows that disadvantages occurring in the use of usual ablative treatment apparatuses, namely relatively long treatment times and relatively high energy input by the laser into the cornea, are reliably avoided. These advantages are in particular achieved by the formation of the eye surgical laser as a photodisruptive laser.

Therein, the laser is suitable to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz.

The treatment apparatus can also comprise a plurality, wherein plurality in particular means at least two, of control devices, which are then in turn formed to perform the method according to the invention. In particular, the control device or the control devices comprises or comprise circuits, for example integrated circuits, processors and further electronic components, to be able to perform the method steps.

In an advantageous form of configuration of the treatment apparatus, the treatment apparatus comprises a storage device for at least temporary storage of at least one control dataset, wherein the control dataset or datasets include(s) control data for positioning and/or focusing individual laser pulses in the cornea, and includes at least one beam device for beam guidance and/or beam shaping and/or beam deflection and/or beam focusing of a laser beam of the laser. Therein, the mentioned control datasets are usually generated based on a measured topography and/or pachymetry and/or morphology of the cornea to be treated and/or the type of the pathologically and/or unnaturally altered area to be removed within the cornea and/or the visual disorder of the eye to be corrected.

Further features and the advantages thereof can be taken from the descriptions of the first inventive aspect, wherein advantageous configurations of each inventive aspect are to be regarded as advantageous configurations of the respectively other inventive aspect.

A third aspect of the invention relates to a computer program including commands, which cause the treatment apparatus according to the second inventive aspect to execute the method steps according to the first inventive aspect. A fourth aspect of the invention relates to a computer-readable medium, on which the computer program according to the third inventive aspect is stored. Further features and the advantages thereof can be taken from the descriptions of the first and second inventive aspects, wherein advantageous configurations of each inventive aspect are to be regarded as advantageous configurations of the respectively other inventive aspect.

Thus, the method according to the invention is in particular a computer-implemented method.

Further features are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

### BRIEF DESCRIPTION OF DRAWINGS

FIG. 1 is a schematic block diagram of an embodiment of a treatment apparatus.

FIG. 2 is a schematic top view to an eye.

FIG. 3 is a further schematic top view to an eye.

FIG. 4 is a still further schematic top view to an eye.

In the figures, identical or functionally identical elements are provided with the same reference characters.

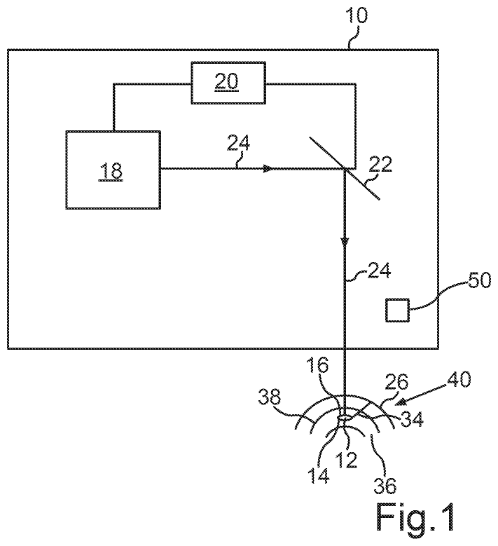

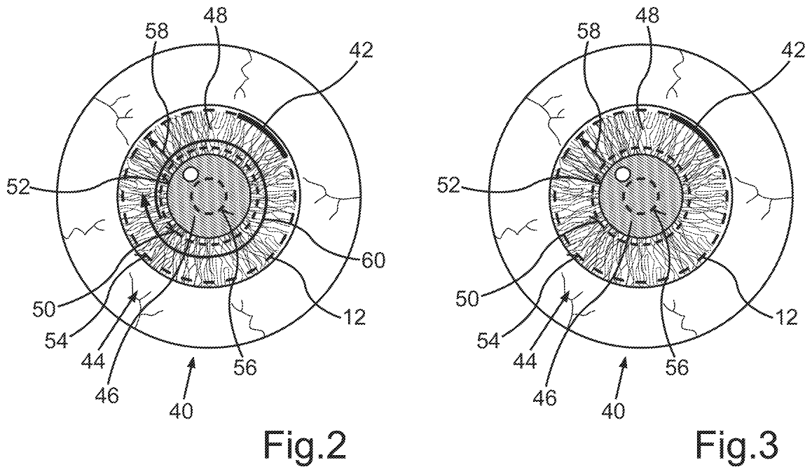

FIG. 1 shows a schematic representation of a treatment apparatus 10 with an eye surgical laser 18 for the separation of a predefined corneal volume or volume body 12 with for example predefined interfaces 14, 16 of a cornea 44 (FIG. 2) of a human or animal eye 40 for example by means of photodisruption. One recognizes that a control device 20 for the laser 18 is formed besides the laser 18, such that it emits pulsed laser pulses in a predefined pattern into the cornea 44 in the present embodiment, wherein the interfaces 14, 16 of the volume body 12 to be separated are generated by the predefined pattern by means of photodisruption. In the illustrated embodiment, the interfaces 14, 16 form a lenticular volume body 12, wherein the position of the volume body 12 is selected in this embodiment such that a pathological and/or unnaturally altered area within a stroma 36 of the cornea 44 or an area, in which visual disorders arise, is encompassed. Furthermore, it is apparent from FIG. 1 that the so-called Bowman's membrane 38 is formed between the stroma 36 and an epithelium.

Furthermore, one recognizes that the laser beam 24 generated by the laser 18 is deflected towards a surface 26 of the cornea by means of a beam deflection device 22, such as for example a scanner. The beam deflection device 22 is also controlled by the control device 20 to generate the mentioned predefined pattern in the cornea. The beam deflection device 22 can for example comprise two mirrors, which are formed for deflecting the impinging laser beam 24. In a neutral position, a so-called 0/0 position of the mirrors, an optical axis of the laser beam 24 is in particular formed.

The illustrated laser 18 is a photodisruptive laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. Alternatively to the treatment apparatus 1 shown in FIG. 1, a method for ablative removal of the volume body 12 can also be used.

In addition, the control device 20 comprises a storage device (not illustrated) for at least temporary storage of at least one control dataset, wherein the control dataset or datasets include(s) control data for positioning and/or for focusing individual laser pulses in the cornea 13. The position data and/or focusing data of the individual laser pulses are generated based on a previously measured topography and/or pachymetry and/or the morphology of the cornea and the pathological and/or unnaturally altered area 32 for example to be removed within the stroma 36 of the eye.

FIG. 2 shows a schematic top view to the eye 40. Further, FIG. 2 shows the volume body 12. Furthermore, an incision 42 is shown, via which the volume body 12 can be removed from the cornea 44.

In the method for controlling the eye surgical laser 18 for the separation of the volume body 12 with the predefined posterior interface 14 and the predefined anterior interface 16 from the human or animal cornea 44, the control of the laser 18 by the control device 20 is effected such that the laser 18 emits laser pulses in a shot sequence into the cornea 44, wherein the interfaces 14, 16 are generated by means of an interaction of the individual laser pulses with the cornea 44, for example by means of photodisruption, by the generation of a plurality of cavitation bubbles along a respective rotation path 60, wherein a respective interface 14, 16 is divided at least into an inner annulus 46 and an outer annulus 48, and wherein the cavitation bubbles are generated along the rotation path 60 from an inner boundary 50 of the outer annulus 48, which faces an outer boundary 52 of the inner annulus 46, to an outer boundary 54 of the outer annulus 48.

In the present embodiment, the outer boundary 52 of the inner annulus 46 corresponds to the inner boundary 50 of the outer annulus 48.

Further, the rotation path 60 is presently spirally formed and a movement direction arrow 58 of the rotation path 60 from the inside to the outside is shown.

In particular, the figures are only schematic and not to scale. The drawings only serve for exemplifying the method. In particular, the inner annulus 46 is formed substantially smaller with respect to the outer annulus 48 than illustrated in the figures.

In a form of configuration, it can be provided that cavitation bubbles are not generated in the inner annulus 48. Herein, it can for example be provided that the inner boundary 56 of the inner annulus 46 is selected with a radius equal to zero. In other words, the inner radius, as presently shown, can be selected such that the inner annulus 46 is formed as a disk. In particular, the radius of the outer boundary 52 of the inner annulus 46 can be selected correspondingly small such that the cavitation bubbles are only generated in the outer annulus 48, and a reliable removal of the volume body 12 can nevertheless be realized.

Further, it can be provided that a respective distance between adjacent cavitation bubbles is increased in the inner annulus 46 with respect to a respective distance between adjacent cavitation bubbles in the outer annulus 48 and/or a respective distance between adjacent rotation path parts of the rotation path 60 is increased in the inner annulus 46 with respect to a respective distance between adjacent rotation path parts of the rotation path 60 in the outer annulus 48. Thus, the distances of the cavitation bubbles in the inner annulus 46 are in particular selected larger than in the outer annulus 48. Herein, it can for example be provided that a repetition rate for emitting the laser pulses is changed for increasing the distance and/or a rotational speed of the beam deflection device 22 for the laser 18 of the treatment apparatus 10 is changed for increasing the distance.

FIG. 3 shows a further schematic top view to an eye 40. In the present embodiment, it is in particular shown that the inner boundary 56 of the inner annulus 46 is selected with a radius greater than zero. Presently, it is in particular to be noted that the illustrated sizes are purely schematically represented. The inner radius of the inner annulus 46 is in particular selected very small such that a reliable removal of the volume body 12 is also realized for example without generating cavitation bubbles further inwards. In other words, the present representation purely schematically serves for explaining the idea of the embodiment according to the invention.

Alternatively thereto, it can in particular be provided that the cavitation bubbles are generated in the inner annulus 46 from the outer boundary 52 of the inner annulus 46 to the inner boundary 56 of the inner annulus 46, and temporally thereafter, the cavitation bubbles are generated from the inner boundary 56 of the inner annulus 46 to the outer boundary 52 of the inner annulus 46. Thus, the cavitation bubbles are generated twice in the inner annulus 46. First, the cavitation bubbles are generated from the outside to the inside and then from the inside to the outside in the inner annulus 46. Herein, it can then for example be provided subsequently thereto that temporally after generating the cavitation bubbles from the inner boundary 56 of the inner annulus 46 to the outer boundary 52 of the inner annulus 46, the cavitation bubbles are generated from the inner boundary 50 of the outer annulus 48 to the outer boundary 54 of the outer annulus 48.

Still alternatively, it can be provided that the cavitation bubbles are generated in the inner annulus 46 from the outer boundary 52 of the inner annulus 46 to the inner boundary 56 of the inner annulus 46, and temporally thereafter, the cavitation bubbles are generated from the inner boundary 50 of the outer annulus 48 to the outer boundary 54 of the outer annulus 48.

Further, it can be provided in an embodiment that the cavitation bubbles are generated from the inner boundary 50 of the outer annulus 48 to the outer boundary 54 of the outer annulus 48 in a first time step, and temporally subsequently, the cavitation bubbles are generated from the inner boundary 56 of the inner annulus 46 to the outer boundary 52 of the inner annulus 46.

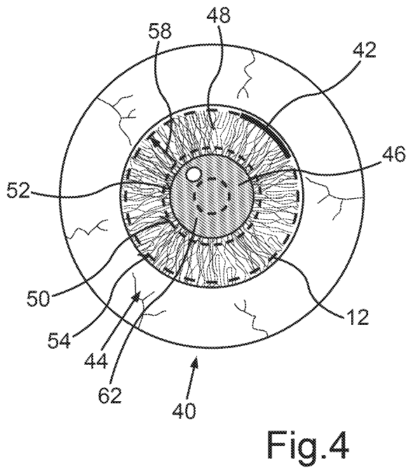

FIG. 4 shows a still further schematic top view to an eye 40. In this embodiment, it can be provided that the respective interfaces 14, 16 are divided at least into one further middle annulus 62. The additional middle annulus 62 is to be purely exemplarily understood. The volume body 12 can also be divided into more than three annuli.

In this embodiment, it is shown that the middle annulus 62 is located between the inner annulus 46 and the outer annulus 48. Then, it can for example be provided that the cavitation bubbles are first generated from the inside to the outside in the outer annulus 48. Subsequently thereto, the cavitation bubbles are generated in the middle annulus 62 from the inside to the outside. Subsequently thereto, the cavitation bubbles are generated in the inner annulus 46 for example also from the inside to the outside.

Thus, it is in particular provided that at least in the outer annulus 48, the cavitation bubbles are always generated from the inner boundary 50 of the outer annulus 48 to the outer boundary 54 of the outer annulus 50. Thus, so-called opaque bubble layers can be prevented.

1. A method for controlling an eye surgical laser for the separation of a volume body with a predefined posterior interface and a predefined anterior interface from a human or animal cornea, comprising:
controlling the laser by means of a control device such that is emits pulsed laser pulses in a shot sequence into the cornea, wherein the predefined posterior and anterior interfaces are generated by means of an interaction of the individual laser pulses with the cornea by the generation of a plurality of cavitation bubbles along a respective rotation path,
wherein a respective interface is divided at least into an inner annulus and an outer annulus, and
wherein the cavitation bubbles are generated along the rotation path from an inner boundary of the outer annulus, which faces an outer boundary of the inner annulus, to an outer boundary of the outer annulus, wherein the outer annulus encompasses the inner annulus.

2. The method according to claim 1, wherein cavitation bubbles are not generated in the inner annulus.

3. The method according to claim 1, wherein an inner boundary of the inner annulus is selected with a radius equal to zero.

4. The method according to claim 1, wherein a respective distance between adjacent cavitation bubbles is increased in the inner annulus with respect to a respective distance between adjacent cavitation bubbles in the outer annulus and/or a respective distance between adjacent rotation path parts of the rotation path is increased in the inner annulus with respect to a respective distance between adjacent rotation path parts of the rotation path in the outer annulus.

5. The method according to claim 1, wherein a repetition rate for emitting the laser pulses is changed for increasing the distance and/or a rotational speed of a deflection device for the laser of the treatment apparatus is changed for increasing the distance.

6. The method according to claim 1, wherein an inner boundary of the inner annulus is selected with a radius greater than zero.

7. The method according to claim 1, wherein the cavitation bubbles are generated in the inner annulus from the outer boundary of the inner annulus to an inner boundary of the inner annulus, and temporally thereafter, the cavitation bubbles are generated from the inner boundary of the inner annulus to the outer boundary of the inner annulus.

8. The method according to claim 5, wherein temporally after generating the cavitation bubbles from the inner boundary of the inner annulus to the outer boundary of the inner annulus, the cavitation bubbles are generated from the inner boundary of the outer annulus to the outer boundary of the outer annulus.

9. The method according to claim 1, wherein the cavitation bubbles are generated in the inner annulus from the outer boundary of the inner annulus to an inner boundary of the inner annulus, and temporally thereafter, the cavitation bubbles are generated from the inner boundary of the outer annulus to the outer boundary of the outer annulus.

10. The method according to claim 1, wherein in a first temporal step, the cavitation bubbles are generated from the inner boundary of the outer annulus to the outer boundary of the outer annulus, and temporally subsequently, the cavitation bubbles are generated from an inner boundary of the inner annulus to the outer boundary of the inner annulus.

11. The method according to claim 1, wherein the respective interfaces are divided at least into an additional middle annulus.

12. The method according to claim 1, wherein the control of the laser is effected such that topographic and/or pachymetric and/or morphologic data of the cornea is taken into account.

13. The method according to claim 1, wherein the control of the laser is effected such that the laser emits laser pulses in a wavelength range between 300 nm and 1400 nm, at a respective pulse duration between 1 fs and 1 ns, and a repetition frequency of greater than 10 kHz.

14. A treatment apparatus with at least one surgical laser for the separation of a volume body with predefined interfaces of a human or animal eye and with at least one control device, which is formed for controlling the laser according to claim 1.

15. The treatment apparatus according to claim 14, wherein the control device
comprises at least one storage device for at least temporary storage of at least one control dataset, wherein the control dataset or datasets include control data for positioning and/or for focusing individual laser pulses in the cornea; and
includes at least one beam device for beam guidance and/or beam shaping and/or beam deflection and/or beam focusing of a laser beam of the laser.

16. A computer program including commands, which cause a treatment apparatus with at least one surgical laser for the separation of a volume body with predefined interfaces of a human or animal eye and with at least one control device formed for controlling the laser to execute the method steps according to claim 1.

17. A computer-readable medium, on which the computer program according to claim 16 is stored.

18. A method for performing a surgical procedure on a human or animal cornea for the separation of a volume body from the cornea,
wherein the interfaces are generated by means of an interaction of the individual laser pulses with the cornea by the generation of a plurality of cavitation bubbles along a respective rotation path,
wherein a respective interface is divided at least into an inner annulus and an outer annulus, and
wherein the cavitation bubbles are generated along the rotation path from an inner boundary of the outer annulus, which faces an outer boundary of the inner annulus, to an outer boundary of the outer annulus, wherein the outer annulus encompasses the inner annulus.

19. The method for performing a surgical procedure according to claim 18, wherein cavitation bubbles are not generated in the inner annulus.

20. The method for performing a surgical procedure according to claim 18, wherein an inner boundary of the inner annulus is selected with a radius equal to zero.

21. The method for performing a surgical procedure according to claim 18, wherein a respective distance between adjacent cavitation bubbles is increased in the inner annulus with respect to a respective distance between adjacent cavitation bubbles in the outer annulus and/or a respective distance between adjacent rotation path parts of the rotation path is increased in the inner annulus with respect to a respective distance between adjacent rotation path parts of the rotation path in the outer annulus.

22. The method for performing a surgical procedure according to claim 18, wherein a repetition rate for emitting the laser pulses is changed for increasing the distance and/or a rotational speed of a deflection device for the laser of the treatment apparatus is changed for increasing the distance.

23. The method for performing a surgical procedure according to claim 18, wherein an inner boundary of the inner annulus is selected with a radius greater than zero.

24. The method for performing a surgical procedure according to claim 18, wherein the cavitation bubbles are generated in the inner annulus from the outer boundary of the inner annulus to an inner boundary of the inner annulus, and temporally thereafter, the cavitation bubbles are generated from the inner boundary of the inner annulus to the outer boundary of the inner annulus.

25. The method for performing a surgical procedure according to claim 24, wherein temporally after generating the cavitation bubbles from the inner boundary of the inner annulus to the outer boundary of the inner annulus, the cavitation bubbles are generated from the inner boundary of the outer annulus to the outer boundary of the outer annulus.

26. The method for performing a surgical procedure according to claim 18, wherein the cavitation bubbles are generated in the inner annulus from the outer boundary of the inner annulus to an inner boundary of the inner annulus, and temporally thereafter, the cavitation bubbles are generated from the inner boundary of the outer annulus to the outer boundary of the outer annulus.

27. The method for performing a surgical procedure according to claim 18, wherein in a first temporal step, the cavitation bubbles are generated from the inner boundary of the outer annulus to the outer boundary of the outer annulus, and temporally subsequently, the cavitation bubbles are generated from an inner boundary of the inner annulus to the outer boundary of the inner annulus.

28. The method for performing a surgical procedure according to claim 18, wherein the respective interfaces (14, 16) are divided at least into an additional middle annulus.
